(19) United States
(12) Patent Application Publication
(10) Pub. No.: US 2022/0192272 A1
(43) Pub. Date: Jun. 23, 2022

(54) PORTABLE ELECTRONIC VAPORIZING DEVICE

(71) Applicant: iKrusher, Inc., Arcadia, CA (US)

(72) Inventor: Ali Kohbodi, Sierra Madre, CA (US)

(21) Appl. No.: 17/124,908

(22) Filed: Dec. 17, 2020

(51) Int. Cl. A24F 40/57; H05B 6/10; H05B 6/06; A24F 40/60

(52) U.S. Cl. CPC A24F 40/57; H05B 6/10; A61M 2205/8206; A24F 40/60; H05B 6/06

(57) ABSTRACT

The presently claimed invention contemplates a device comprising a filter configured to direct a vaporized material to an outlet, a housing configured to removably couple to the filter, an induction assembly. The housing comprises a control interface, wherein the control interface receives a user input. The housing further comprises a control module, wherein the control module processes at least one of the user input and a sensor input. The present invention contemplates that the control module can control an execution of a heating protocol in response at least the user input and the sensor input. The induction assembly further comprises an induction element configured to produce an electromagnetic field according to a heating protocol and an inductive element within a threshold distance of the electromagnetic field that is configured to at least partially convert electromagnetic radiation into heat.

### FIELD OF THE INVENTION

The field of the invention is a portable electronic vaporizer that uses induction heating.

### BACKGROUND

The background description includes information that may be useful in understanding the present invention. It is not an admission that any of the information provided herein is prior art or relevant to the presently claimed invention, or that any publication specifically or implicitly referenced is prior art.

Various vaporizing technologies are known. For example, U.S. Pat. No. 10,058,129 to Monsees describes a cartridge-based vaporizer using a heater that includes a first plate, a second plate, a wick, and a resistive heating element in contact with the first plate and the second plate.

As another example, U.S. Patent Publication No. 10,517,334 to Volodarsky describes a vaporizer having an atomizer that uses a thermally conductive element. Volodarsky also describes an embodiment using thermal radiation, in which light is used to heat the substance. Lastly, Volodarsky also described an embodiment using convective technology, in which hot air is passed over a substance.

In contrast to the prior art, the present invention describes system in which induction technology is used to heat an inductive element. By using induction technology to vaporize material, the present invention provides solutions to the shortcomings of non-inductive heating technologies. Advantages of inductive technologies include, but are not limited to, precise temperature control, easily exchangeable inductive containers for multiple consecutive uses, and speed of heating. As such, the present invention improves the speed and accuracy of heating over conventional vaporizers, which is especially important when volatilizing materials using heat. Applying the appropriate amount of heat based on the type of material with the accuracy of induction heating can improve user safety by reducing the formation of unwanted byproducts, such as smoke and associated carcinogens.

While vaporizers with heating elements are known in the art, there is still a need for improved devices that deliver heat in a precise and quick manner.

These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### SUMMARY OF THE INVENTION

The inventive subject matter provides a device comprising a filter configured to direct a vaporized material to an outlet, a housing configured to removably couple to the filter, an induction assembly.

The housing can comprise a control interface, wherein the control interface receives a user input. The housing further comprises a control module, wherein the control module processes at least one of the user input and a sensor input.

The present invention contemplates that the control module can control an execution of a heating protocol in response at least the user input and the sensor input. The induction assembly further comprises an induction element configured to produce an electromagnetic field according to a heating protocol and an inductive element within a threshold distance of the electromagnetic field that is configured to at least partially convert electromagnetic radiation into heat.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A depicts a cross-sectional view of an embodiment of a vaporizing device.

FIG. 1B depicts an anterior view of the vaporizing device of FIG. 1A.

FIG. 2A depicts an exploded view of the vaporizer device of FIG. 1A.

FIG. 2B depicts a cross-sectional view of the vaporizing device of FIG. 1A.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Also, as used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

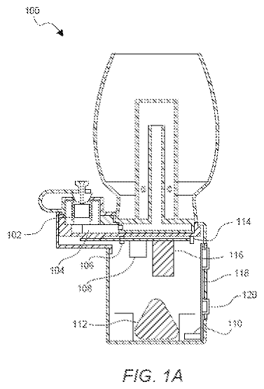

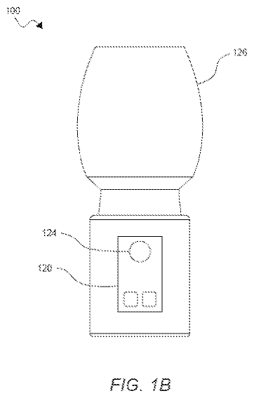

FIG. 1A depicts a cross-sectional view of a vaporizing device 100. Vaporizing device 100 is depicted as including, but is not limited to including, induction element 102, vapor path 104, control module 106, resistor 108, power conduit 110, battery pack 112, indicator 114, capacitor 116, input module 118, and control interface 120.

Induction element 102 is a type of electrical transformer that uses a fluctuating magnetic field to induce voltage across a coil. For example, induction element 102. Induction element 102 is configured to send electromagnetic radiation to an inductive material to be converted to thermal energy. Inductive material can include metal components with high ferrous metal content, such as iron and magnetic grade stainless steels.

Vapor path 104 is an opening sized and dimensioned to allow the flow of gases and particulates suspended therein through from a first opening to a second opening. In preferred embodiments, vapor path 104 is coupled to a heating element at a first end and coupled to a filter pathway configured to filter the gases and particulates. For example, vapor path 104 can separate an inductive heating element that volatilizes a substance into a heated vapor and a water filter pathway that percolates the heated vapor through water to reduce the temperature and filter out undesirable elements, such as ash.

Control module 106 may include any one or more control components configured to control the flow of signals to one or more coupled electronic components. In one embodiment, control module 106 is a printed circuit board configured to control the operation of any one or more electronic components. For example, control module 106 can include integrated circuits configured to transmit one or more electrical signals produced via user inputs to cause power to be delivered to an inductive heating element. In another example, control module 106 may include integrated circuits configures to transmit one or more electrical signals to a timing module.

In some embodiments, control module 106 may include multiple sub-units. For example, control module 106 may include a collection of operatively coupled modules, such as timing modules, heating modules, user interface modules, and/or the like. Control module 106 may be configured to send one or more electrical signals to any one or more receivers configured to process incoming signals.

Control module 106 may also be coupled to one or more memory storage devices configured to store one or more program instructions. For example, memory storage devices can include FLASH memory that stores program instructions, such as heating protocols for induction element 102, lighting protocols, timing protocols, and any other type of storable program instructions. It is further contemplated that control module 106 can change one or more stored program instruction in response to an external input. For example, control module 106 can be configured to manage the installation and execution of a software update from a manufacturer. In another example, control module 106 can be configured to alter existing program instructions in response to user input, such as heating preferences, lighting effects, and/or the like.

In some embodiments, control module 106 may be operatively coupled to other devices using a wireless connection. For example, control module 106 may send wireless signals, including, but not limited to, radio transmissions, infrared transmissions, microwave transmissions, and light wave transmissions.

In one embodiment, control module 106 is operatively coupled to a heating element. For example, control module 106 may be operatively coupled to an induction heating element.

In other embodiments, control module 106 is operatively coupled to one or more modules configured to control the operation of vaporizer 100. For example, control module 106 may be configured to receive one or more user inputs causing control module 106 to send an electrical signal to a lighting module, thereby controlling the timing, colors, and brightness of one or more lights.

In some embodiments, control module 106 may include one or more processor. For example, control module 106 may include one or more microprocessors, microcontrollers, and embedded processors to control how one or more functions of vaporizer 100 are executed. In a related example, control module 106 may use one or more processors to dynamically adjust how a material is heated based on one or more variables, such as ambient temperature, amount of material, type of material, and/or status of power sources.

Resistor 108 is a device having a designated resistance for the passage of electric signals enabling the transmission of electric signals from control module 106 to indicator 114 and other electronic components. In some embodiments, vaporizer 100 does not include resistor 108.

Power conduit 110 can include any means of connecting vaporizer 100 to an external power source. For example, power conduit 110 can be a USB-C female receptacle connector capable of enabling the flow of electricity to one or more components of vaporizer 100. In some embodiments, power conduit 110 can additionally or alternatively include wireless means of connecting vaporizer 100 to an external power source. For example, power conduit 110 can include a receive coil in an inductive system for wirelessly transmitting power to vaporizer 100.

Power source 112 can include any source of electricity used to operate vaporizer 100. In preferred embodiments, power source 112 is a power storage medium. For example, power source 112 may be a set of batteries. As used herein, batteries may include, but are not limited to, disposable, replaceable and/or rechargeable batteries. For example, batteries may include any one or more of an alkaline, lead acid battery, or a lithium-ion battery. Power source 112 is electrically configured to deliver the voltage to power one or more components of vaporizer 100, such as indicator 114.

In another embodiment, power source 112 includes an electrical conduit physically coupled to a grounded outlet at a first end and to vaporizer 100 at a second end. For example, power source 112 can be a conventional plug that is physically coupled to an electrical outlet.

In yet another embodiment, power source 112 can include a portable power source (e.g., battery) and a substantially fixed power sources (e.g., conventional electrical outlets). For example, power source 112 can include a physical electrical conduit, such as a USB-C cable, and a rechargeable battery. In such embodiments, the present invention can be configured to draw power from any one or more power sources in combination or individually.

Indicator 114 can include any one or more means for alerting a user. In a preferred embodiment, indicator 114 includes one or more lights, such as light-emitting diodes (LED), that are configured to activate in response to one or more input signals. For example, indicator 114 can include an RGB LED light that is configured to emit the color red in response to induction element 102 reaching a temperature of 400-500° C. Indicator 114 can be configured to emit lights of various colors and brightness. In some embodiments, indicator 114 can be operatively coupled to one or more elements associated with control module 106. For example, indicator 114 can be an LED light coupled to a microcontroller, wherein the microcontroller can receive user inputs and send program instructions to change the color and brightness of the LED light according to the user inputs.

In one embodiment indicator 114 is electrically coupled to a heating element to display colors based on the temperature range reached.

In another embodiment, indicator 114 is electrically coupled to the user control inputs to process one or more user inputs. For example, indicator 114 can be configured to display a rainbow effect when transitioning from the default display mode to another mode. As another example, indicator 114 can be configured to display white while in a default display mode. Indicator 114 can be configured to display a red heating icon providing indication that the vaporizing device 100 is in the process of heating. In another example, indicator 114 is configured to display green once the requisite heating temperature is reached. Indicator 114 is configured to display a logo and “Power ON” sign when the power button is pressed.

Capacitor 116 is a device that stores electrical energy and is composed of at least two electrical conductors separated by an insulator. In some embodiments, vaporizer 100 does not include capacitor 116.

Input module 118 is a printed circuit board configured to receive input from the device controls such as the control interface 120.

Control interface 120 can be any means for translating user input into one or more signals managed by control module 106. In one embodiment, control interface 120 is a button pad configured to receive input controlling the behavior of the vaporizer, such as heating and timing characteristics, based on the user's selection of one or more operations parameters, including, but not limited to, a material to be vaporized, user-customized heating preferences, and/or the like.

Once control interface 120 receives input on the desired mode, control interface 120 is configured to cause control module 106 to cause induction element 102 to heat up in a manner prescribed by the desired mode. For example, control interface 120 can receive an input of dry herb as the material, which causes control module 106 to execute a heating protocol at a temperature range and timing that increases the efficiency of vaporization.

FIG. 1B depicts an anterior view of a vaporizing device 100. As depicted, vaporizing device 100 further includes input element 124 operatively coupled to control interface 120 and filter 126. Control interface 120 can include a display associated with the operation of vaporizer 100.

Control interface 120 can include any one or more elements that can individually and/or cooperatively control the operation of vaporizer 100. For example, control interface 120 can include a screen that displays one or more temperature options to a user via a software user interface. In another example, control interface 120 can include one or more haptic feedback mechanisms to allow a user to operate vaporizer 100 without a direct line of sight. In yet another example, control interface 120 can include a microphone to detect one or more user voice commands. In a final illustrative example, control interface 120 can include a speaker to communicate information to a user, such as an alert when an inductive element reaches a target temperature.

In some embodiments, control interface 120 may include one or more physical inputs, such as button 124. It is contemplated that button 124 can include any one or more means of translating a user input into a signal. For example, button 124 can translate a user input of five presses of button 124 to turn off vaporizer 100. In one embodiment, button 124 can be a transducer configured to change physical pressure on a button to an electrical signal using one or more electromechanical mechanisms, such as transducers. For example, button 124 can extend the duration of heating time of an induction heating element.

Transducers can include any means by which one form of energy can be converted to another. For example, transducers can include, but are not limited to, pressure transducers, inductive transducers, displacement transducers, oscillator transducers, photovoltaic transducers, piezoelectric transducers, electromagnetic transducers, and Hall effect transducers. Transducers can also include active transducers or passive transducers.

In other embodiment, button 124 is activated using alternative and/or additional means. For example, button 124 can be activated via a user input into a capacitive touch sensor operatively coupled to button 124. It is further contemplated that button 124 can be operatively coupled to one or more computer implemented program instructions, such as software that can offer additional customizability and functionality associated with a user input.

In some embodiments, vaporizer 100 includes one or more computing components, including, but not limited to, a computer processor, a volatile memory storage, a non-volatile memory storage, and an input/output interface. For example, vaporizer 100 can include a propriety system on a chip (SoC) to execute one or more computer implemented program instructions in response to one or more inputs. Inputs can be received from any one or more sensors. In one example, sensors can include electromechanical mechanisms actuated via intentional user input, such as piezoelectric mechanisms. In another example, sensors can include mechanisms that do not require intentional user input, such as light sensors, temperature sensors, accelerometers, sound sensors, and/or the like.

Filter 126 can include any means by which vaporized material is delivered to an outlet. In the depicted embodiment, filter 126 is positioned proximate to the vapor path 104 to allow the flow of vapor through vapor path 104 to an opening of filter 126.

In preferred embodiments, filter 126 includes a lumen that is physically configured to at least partially filter vaporized material and gases through a liquid layer. For example, filter 126 can be a water filter that percolates vaporized material and gases through water via a filtering pathway.

In other embodiments, filter 126 comprises a lumen that is configured to increase the distance between a source of heated vapor to an outlet, such as a mouthpiece. For example, filter 126 can be a coiled tube that increases the physical distance that vaporized material travels to reach an outlet.

In one embodiment, filter 126 includes a gasket configured to seal a connection between an opening of filter 126 and engagement mechanism 202, discussed in further detail below, of vaporizing device 100. For example, filter 126 can include a detachable gasket that uses a resilient material to secure filter 126 to engagement mechanism 202, thereby creating a sealed connection with housing 206 of vaporizer 100.

Filter 126 can be secured to housing 206 using engagement mechanism 202. via any mechanism that can create a substantially sealed fit between filter 126 and housing 206. For example, filter 126 can secure onto housing 206 using any one or more of a screw fit, a snap fit, a pressure-based fit, and a magnet-based fit.

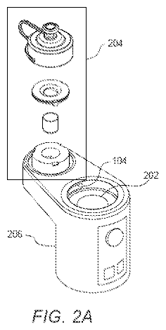

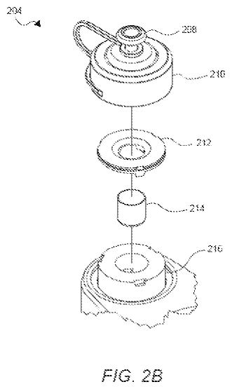

FIG. 2A depicts an exploded view of vaporizer device 100. Vaporizer device 100 further includes vapor path 104, engagement mechanism 202, heating assembly 204, and housing 206.

In the depicted embodiment, induction assembly 204 includes lid 208, lid assembly 210, lid assembly holder 212, inductive cup 214, and induction assembly 216. Induction assembly 204 will be described in further detail in the description of FIG. 2B below.

Engagement mechanism 202 can include any mechanism that can create a substantially sealed fit between filter 126 and housing 206. For example, engagement mechanism 202 can be sized and configured to allow any one or more of a screw fit, a snap fit, a pressure-based fit, and a magnet-based fit between filter 126 and housing 206.

Housing 206 can be a housing that includes any one or more operatively coupled components associated with the operation of vaporizer 100. Housing 206 can be made from any one or more materials. Materials can include, but are not limited to, plastics, metals, woods, ceramics, glass, composite materials, and resilient materials (i.e., silicone and rubbers).

In some embodiments, housing 206 includes multiple physically coupled components. For example, housing 206 can include a substantially rigid acrylonitrile butadiene styrene housing with a removable silicone sleeve configured to add resilient characteristics to the device.

Vapor pathway 104 is an opening sized and dimensioned to allow the flow of gases and particulates suspended therein through from a first opening to a second opening. In preferred embodiments, vapor pathway 104 is coupled to a heating element at a first end and coupled to a filter pathway configured to filter the gases and particulates. For example, vapor path 104 can separate an inductive heating element that volatilizes a substance into a heated vapor and a water filter pathway that percolates the heated vapor through water to reduce the temperature and filter out undesirable elements, such as ash.

FIG. 2B depicts a cross-sectional view of vaporizing device 100.

As depicted, induction assembly 204 includes lid 208, lid seating 210, cover 212, inductive element 214, and induction assembly 216.

Lid 208 can be any mechanism by which air inflow to vaporizer 100 is regulated. In the depicted embodiment, lid 208 is a removable physical barrier that can be switched from a substantially sealed to a substantially unsealed state, or vice versa, to control the inflow of air in and through housing 206.

In some embodiments, lid 208 can be an electromechanical mechanism configured to automatically close and open in response to a change in one or more variables. For example, lid 208 can be operatively coupled to an anemometer in housing 206, which can cause lid 208 to substantially cut off air intake by changing a physical configuration of lid 208.

However, the present invention contemplates the use of any mechanism along the airflow path configured to substantially prevent airflow through housing 206. For example, housing 206 can include an actuator assembly configured to impede airflow in response to a trigger, such as a detected period of non-use, by physically sealing off vapor pathway 104.

Lid seating 210 can include any one or more mechanisms configured to removably couple with lid 208. In preferred embodiments, lid assembly holder 210 is at least partially made of an insulating material. For example, lid assembly holder 210 can be made of glass. In another example, lid assembly holder 210 can be made of ceramic. In yet another example, lid assembly holder 210 can be made of silicone.

Lid 208 and lid seating 210 can removably mate using any mechanism. For example, lid 208 and lid seating 210 can removable mate using any one or more of a screw mechanism, a snap fit mechanism, a pressure-based fit mechanism, and a magnetic attachment mechanism.

Cover 212 is a unit configured to removably couple and substantially cover induction element 216 when cover 212 is coupled to induction element 216. Cover 212 and induction element 216 can removably couple in any manner known in the art, including through physical and/or magnetic means. In the depicted embodiment, cover 212 is further configured to removably couple with lid seating 210.

Cover 212 can be made of any one or more materials. For example, cover 212 can be made of any one or more of a ceramic material, a metal, a plastic, a glass, a silicone-based material, and/or the like. In preferred embodiments, cover 212 is made of materials that are configured to withstand the maximum heat output of vaporizer 100 as determined by the configuration of inductive element 214 and induction element 216.

Inductive element 214 can include any inductive materials that are sized and shaped to transfer heat via inductive technologies to a material. In the depicted embodiment, inductive element 214 is a cup that is sized and dimensioned to be placed between induction element 216 and cover 212.

The present invention advantageously contemplates the use of substantially non-porous materials in inductive element 214. The use of non-porous materials advantageously reduces the adherence of the heated material to the increased surface area of substantially porous surfaces. For example, inductive element 214 can be made of an inductive alloy that defines a substantially smooth lumen having substantially non-porous inner walls. In another example, inductive element 214 can be made of a combination of inductive and non-inductive materials, such as a metal cup made of induction-grade stainless steel that is enameled using a powdered glass.

Inductive element can be composed of materials including, but not limited to, magnetic grade stainless steels and other metal components with high ferrous metal content. Inductive element 214 is configured to allow for precise temperature control, thereby allowing for an equal, consistent, temperature throughout the chamber. By enabling highly precise temperature control, vaporizer 100 can adapt heating protocols (e.g., timing and temperatures) to adapt to the material to be heated and at least partially vaporized. The use of inductive element 214 also reduces hot spots, which increases temperature consistency and reduces inaccurate temperature readings.

Induction element 216 includes any one or more elements configured to cause inductive element 214 to increase in temperature. For example, induction element 216 can include an induction coil configured to create an electromagnetic field in response to the flow of an alternative current through the induction coil.

Induction element 216 receives a flow of electricity to heat inductive element 214 in a manner determined by control interface 120 and control module 106 in response to one or more inputs and/or variables. For example, control interface 120 can receive and send user inputs that cause control module 106 to enable the flow of an alternating current to induction element 216 until a designated temperature is reached.

In some embodiments, control module 106 can receive inputs from one or more sensors, such as temperature sensors, in order to control the manner in which inductive element 214 is heated. For example, control module 106 can receive temperature readings in 1 millisecond increments to ensure that the temperature of inductive element 214 stays consistent. In a related example, control module 106 can cause one or more processors to execute a heating protocol that automatically heats inductive element 214 in accordance with a particular heating protocol for a particular material, such as a solventless concentrate of a plant material.

Induction element 216 can be configured to receive user-customized inputs. For example, induction element 216 can receive a user-created heating protocol that causes inductive element to be heated for an ordered series of temperatures, each with a specific heating duration.

In preferred embodiments, induction element 216 is capable of heating substances including but not limited to tobacco, cannabis, lavender, chamomile, and various plant materials. In preferred embodiments, induction element 216 is configured to heat up the selected substance within a predetermined period of time. For example, if one or more user inputs triggers the heating of plant materials, induction element 216 can be limited to an available temperature range of 240° F. to 430° F. (115° C. to 221° C.) and the session duration will range from 2 minutes to 5 minutes. In another example, if one or more user inputs triggers the heating of concentrated oil-based extracts, induction element 216 can be limited to an available temperature range of 450° F. to 710° F. (230° C. to 377° C.). Induction element 216 can also be limited to a session duration from a range of 20 seconds to 1 minute. In some examples, the session duration can be dependent on one or more session variable. Session variables can include but are not limited to, a type of material being heated, a desired user vaporizing experience, a type of power source, an availability of power, and a safety-related restriction In this way, the present invention advantageously allows for material specific adjustments to be made to the manner in which a particular material is heated.

In some embodiments, induction element 216 is removable from vaporizing device 100. In other embodiments, induction element 216 is electrically coupled with indicator 114 to communicate information regarding a heating cycle. For example, indicator 114 can display colors based on the temperature range reached in the heat chamber. In another example, indicator 114 can provide tactile feedback to a user in the form of vibration or other haptic feedback mechanisms. In yet another example, indicator 114 can provide auditory feedback by causing a voice prompt to communicate that inductive element 214 has reached the target temperature.

In the depicted embodiment, induction element 216 is advantageously positioned substantially laterally from filter 126. For example, induction element 216 can be positioned in a separate lateral portion of housing 206, such that inductive element 214 and induction element 216 are separately accessible. In such embodiments, it is contemplated that the physical separation of the inductive elements of vaporizer advantageously allows a user to access inductive element 214 directly by removing cover 212. For example, a user can remove a first inductive element, such as a small cup made of a ferrous material, with a second inductive element that is similarly sized and dimensioned. In this way, a user can quickly exchange inductive cups containing spent material with replacement inductive cups containing unspent material. Laterally separating inductive element 214 and induction element 216 from filter 126 also advantageously allows a user to access inductive element 214 without requiring removal of filter 126. In conventional stacked arrangements, heating elements can be positioned below a filter with a mouthpiece, which requires the removal of the filter to access the spent material in the heating elements.

In another embodiment, inductive element 214 is configured to run a cleaning protocol without having to removing the inductive cups from vaporizer 100. For example, induction element 216 can be configured to receive user input from control interface 120 that designates a high temperature mode. Control module 106 can execute a high temperature mode that will clear additional residue remaining in inductive element 214 to reduce the amount of remnant materials within an interior volume of the inductive cup. During the cleaning protocol, vaporizer 100 can include one or more notifications, such as a warning reminding the user not to use the device and a “cleaning” status notification.

Additionally, the present invention also increases the physical separation one or more electrical and inductive components from filter 126, which can increase safety in embodiments where filter 126 contains a liquid.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A device for heating and at least partially vaporizing a material, comprising:
a filter configured to direct a vaporized material to an outlet;
a housing configured to removably couple to the filter, wherein the housing further comprises;
a control interface, wherein the control interface receives a user input;
a control module,
wherein the control module processes at least one of the user input and a sensor input, and
wherein the control module controls an execution of a heating protocol in response the at least of the user input and the sensor input;
an induction assembly is separated and positioned substantially laterally to the filter, wherein the induction assembly further comprises;
an induction element configured to produce an electromagnetic field according to the heating protocol;
an inductive element within a threshold distance of the electromagnetic field, wherein the inductive element produces heat in response the electromagnetic field.

2. The device of claim 1, wherein the control interface further comprises a display.

3. The device of claim 2, wherein the display is configured to transmit visual information associated with a temperature of the inductive element.

4. The device of claim 1, wherein the user input is at least one of an electrical input and a physical input.

5. The device of claim 4, wherein an electromechanical mechanism transduces the physical input into an electrical signal.

6. The device of claim 1, wherein the induction assembly further comprises a cover, wherein the cover at least partially encloses the inductive element between the induction element and the cover in a closed configuration.

7. The device of claim 1, further comprising an indicator.

8. The device of claim 7, wherein the indicator emits light.

9. The device of claim 7, wherein the indicator emits sounds.

10. The device of claim 7, wherein the indicator emits tactile feedback.

11. The device of claim 1, wherein the user input is a modification to the heating protocol.

12. The device of claim 1, wherein the inductive element is substantially non-porous.

13. The device of claim 1, wherein the control module is operatively coupled to at least one sensor.

14. The device of claim 13, wherein the at least one sensor is a temperature sensor.

15. The device of claim 13, wherein the at least one sensor is a capacitive touch sensor.

16. The device of claim 13, wherein the at least one sensor is a microphone.

17. The device of claim 13, wherein the at least one sensor is a pressure.

18. The device of claim 1, wherein the control module is operatively coupled to a wireless communication module.

19. The device of claim 18, wherein the wireless communication module uses a communications protocol selected from the group consisting of: a radio transmission, an infrared transmission, a microwave transmission, and a light wave transmission.
